(19) United States
(12) Patent Application Publication
(10) Pub. No.: US 2003/0162722 A1
(43) Pub. Date: Aug. 28, 2003

(54) Pharmaceutical compositions comprising acryloyl distamycin derivatives and topoisomerase I and II inhibitors

(76) Inventors: Maria Cristina Geroni, Milano (IT); Paolo Cozzi, Milano (IT); Italo Beria, Milano (IT)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/297,915

(22) PCT Filed: Jun. 20, 2001
(86) PCT No.: PCT/EP01/07059

(30) Foreign Application Priority Data
Jun. 23, 2000 (GB) 0015444.3

(51) Int. Cl. A61K 38/06; A61K 31/7048; A61K 31/704; A61K 31/403

(52) U.S. Cl. 514/18; 514/34; 514/27; 514/411; 514/297; 514/680

(57) ABSTRACT

The present invention provides the combined use of acryloyl distamycin derivatives, in particular α-bromo- and α-chloro-acryloyl distamycin derivatives of formula (I), as set forth in the specification, and an antineoplastic topoisomerase I or II inhibitor, in the treatment of tumors. Also provided is the use of the said combinations in the treatment or prevention of metastasis or in the treatment of tumors by inhibition of angiogenesis.

The present invention relates to the field of cancer treatment and provides an antitumor composition comprising a substituted acryloyl distamycin derivative, more particularly an α-bromo- or α-chloro-acryloyl distamycin derivative, and a topoisomerase inhibitor of type I or II, having a synergistic antineoplastic effect.

Distamycin A and analogues thereof, hereinafter referred to as distamycin and distamycin-like derivatives, are known in the art as cytotoxic agents useful in antitumor therapy.

Distamycin A is an antibiotic substance with antiviral and antiprotozoal activity, having a polypyrrole framework [Nature 203: 1064 (1964); J. Med. Chem. 32: 774-778 (1989)]. The international patent applications WO 90/11277, WO 98/04524, WO 98/21202, WO 99/50265, WO 99/50266 and WO 01/40181 (claiming priority from British patent application No. 9928703.9), all in the name of the applicant itself and herewith incorporated by reference, disclose acryloyl distamycin derivatives wherein the amidino moiety of distamycin is optionally replaced by nitrogen-containing ending groups such as, for instance, cyanamidino, N-methylamidino, guanidino, carbamoyl, amidoxime, cyano and the like, and/or wherein the polypyrrole framework of distamycin, or part of it, is replaced by varying carbocyclic or heterocyclic moieties.

The present invention provides, in a first aspect, a pharmaceutical composition for use in antineoplastic therapy in mammals, including humans, comprising a pharmaceutically acceptable carrier or excipient;

an acryloyl distamycin derivative of formula (I):

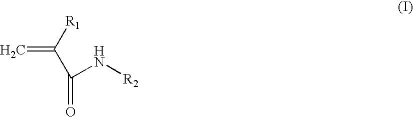

wherein:

R₁ is a bromine or chlorine atom;

R₂ is a distamycin or distamycin-like framework; or a pharmaceutically acceptable salt thereof; and

an antineoplastic topoisomerase inhibitor of type I or II.

The present invention includes, within its scope, the pharmaceutical compositions comprising any of the possible isomers covered by the compounds of formula (I), both considered separately or in admixture, as well as the metabolites and the pharmaceutically acceptable bio-precursors (otherwise known as pro-drugs) of the compounds of formula (I).

In the present description, unless otherwise specified, with the term distamycin or distamycin-like framework R₂ we intend any moiety structurally closely related to distamycin itself, for instance by optionally replacing the ending amidino moiety of distamycin and/or its polypyrrole framework, or part of it.

Topoisomerase I and II inhibitors are known in the art as described in various scientific publications.

The main representatives for topoisomerase I inhibitors are camptothecin derivatives such as, for instance, CPT-11, Topotecan, 9-amino-camptothecin, 9-nitro-camptothecin and 10,11-methylenedioxy-camptothecin.

Among the topoisomerase II inhibitors are, in particular, the anthracycline derivatives such as doxorubicin, daunorubicin, epirubicin, nemorubicin and idarubicin; the podophyllotoxin compounds etoposide and teniposide; the anthraquinone derivative like mitoxantrone and losoxantrone; the acridine derivatives like amsacrine and actinomaycin D. See, for a reference, Cancer, Principles and Practice of Oncology, Lippincott-Raven Ed. (1997), 452-467.

According to a preferred embodiment of the invention, herewith provided are the above pharmaceutical compositions wherein the topoisomerase inhibitors are topoisomerase II inhibitors, in particular doxorubicin and etoposide.

According to another preferred embodiment of the invention, herewith provided are the above pharmaceutical compositions wherein, within the acryloyl distamycin derivative of formula (I), R₁ has the above reported meanings and R₂ is a group of formula (II) below:

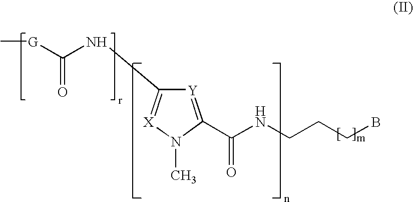

wherein

m is an integer from 0 to 2;

n is an integer from 2 to 5;

r is 0 or 1;

X and Y are, the same or different and independently for each heterocyclic ring, a nitrogen atom or a CH group;

G is phenylene, a 5 or 6 membered saturated or unsaturated heterocyclic ring with from 1 to 3 heteroatoms selected among N, 0 or S, or it is a group of formula (III) below:

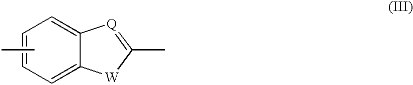

wherein Q is a nitrogen atom or a CH group and W is an oxygen or sulfur atom or it is a group NR₃ wherein R₃ is hydrogen or C₁-C₄ alkyl;

B is selected from the group consisting of

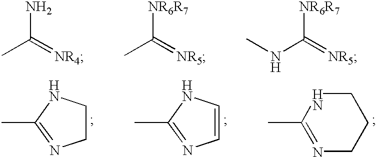

—CN; —NR₅R₆; —CONR₅R₆; —NHCONR₅R₆

wherein R₄ is cyano, amino, hydroxy or C₁-C₄ alkoxy; R₅, R₆ and R₇, the same or different, are hydrogen or C₁-C₄ alkyl.

In the present description, unless otherwise specified, with the term C₁-C₄ alkyl or alkoxy group we intend a straight or branched group selected from methyl, ethyl, n-propyl, isopropyl, n-butyl, isobutyl, sec-butyl, tert-butyl, methoxy, ethoxy, n-propoxy, isopropoxy, n-butoxy, isobutoxy, sec-butoxy or tert-butoxy.

Even more preferred are the pharmaceutical compositions of the invention comprising the above acryloyl distamycin derivative of formula (I) wherein R₁ is bromine or chlorine; R₂ is the above group of formula (II) wherein r is 0, m is 0 or 1, n is 4 and B has the above reported meanings.

Still more preferred, within this class, are the pharmaceutical compositions comprising the compounds of formula (I) wherein R₁ is bromine or chlorine; R₂ is the above group of formula (II) wherein r is 0, m is 0 or 1, n is 4, X and Y are both CH groups and B is selected from:

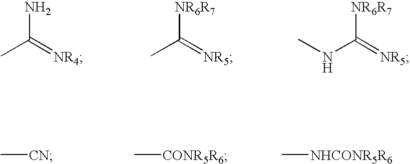

—CN; —CONR₅R₆; —NHCONR₅R₆

wherein R₄ is cyano or hydroxy and R₅, R₆ and R₇, the same or different, are hydrogen or C₁-C₄ alkyl.

Pharmaceutically acceptable salts of the compounds of formula (I) are those with pharmaceutically acceptable inorganic or organic acids such as, for instance, hydrochloric, hydrobromic, sulfuric, nitric, acetic, propionic, succinic, malonic, citric, tartaric, methanesulfonic, p-toluenesulfonic acid and the like.

Examples of preferred acryloyl distamycin derivatives of formula (I), within the compositions object of the invention, optionally in the form of pharmaceutically acceptable salts, preferably with hydrochloric acid, are:

1. N-(5-{[(5-{[(5-{[(2-{[amino(imino)methyl]amino}ethyl)amino]carbonyl}-1-methyl-1H-pyrrol-3-yl)amino]carbonyl}-1-methyl-1H-pyrrol-3-yl)amino]carbonyl}-1-methyl-1H-pyrrol-3-yl)-4-[(2-bromoacryloyl)amino]-1-methyl-1H-pyrrole-2-carboxamide hydrochloride;

2. N-(5-{[(5-{[(5-{[(2-{[amino(imino)methyl]amino}propyl)amino]carbonyl}-1-methyl-i H-pyrrol-3-yl)amino]carbonyl}-1-methyl-i H-pyrrol-3-yl)amino]carbonyl}-1-methyl-1H-pyrrol-3-yl)-4-[(2-bromoacryloyl)amino]-1-methyl-1H-pyrrole-2-carboxamide hydrochloride;

3. N-(5-{[(5-{[(5-{[(3-amino-3-iminopropyl) amino]carbonyl}-1-methyl-1H-pyrrol-3-yl)amino]carbonyl}-1-methyl-1H-pyrrol-3-yl)amino]carbonyl}-1-methyl-1H-pyrrol-3-yl)-4-[(2-bromoacryloyl)amino]-1-methyl-1H-pyrrole-2-carboxamide hydrochloride;

4. N-(5-{[(5-{[(5-{[(3-amino-3-iminopropyl)amino]carbonyl}-1-methyl-1H-pyrrol-3-yl)amino]carbonyl}-1-methyl-1H-pyrrol-3-yl)amino]carbonyl}-1-methyl-1H-pyrrol-3-yl)-4-[(2-bromoacryloyl)amino]-1-methyl-1H-imidazole-2-carboxamide hydrochloride;

5. N-(5-{[(5-{[(5-{[(3-amino-3-iminopropyl)amino]carbonyl}-1-methyl-1H-pyrrol-3-yl)amino]carbonyl}-1-methyl-1H-pyrrol-3-yl)amino]carbonyl}-1-methyl-1H-pyrrol-3-yl)-3-[(2-bromoacryloyl)amino]-1-methyl-1H-pyrazole-5-carboxamide hydrochloride;

6. N-(5-{[(5-{[(5-{[(3-amino-3-oxopropyl)amino]carbonyl}-1-methyl-1H-pyrrol-3-yl)amino]carbonyl}-1-methyl-1H-pyrrol-3-yl)amino]carbonyl}-1-methyl-1H-pyrrol-3-yl)-3-[(2-bromoacryloyl)amino]-1-methyl-1H-pyrazole-5-carboxamide;

7. N-(5-{[(5-{[(5-{[(2-{[amino(imino)methyl]amino}ethyl)amino]carbonyl}-1-methyl-1H-pyrrol-3-yl)amino]carbonyl}-1-methyl-1H-pyrrol-3-yl)amino]carbonyl}-1-methyl-1H-pyrrol-3-yl)-4-[(2-chloroacryloyl)amino]-1-methyl-1H-pyrrole-2-carboxamide hydrochloride;

8. N-(5-{[(5-{[(3-{[amino(imino)methyl]amino}propyl)amino]carbonyl}-1-methyl-1H-pyrrol-3-yl)amino]carbonyl}-1-methyl-1H-pyrrol-3-yl)-4-[(2-bromoacryloyl)amino]-1-methyl-1H-pyrrole-2-carboxamide hydrochloride;

9. N-(5-{[(5-{[(3-amino-3-iminopropyl)amino]carbonyl}-1-methyl-1H-pyrrol-3-yl)amino]carbonyl}-1-methyl-1H-pyrrol-3-yl)-4-[(2-bromoacryloyl)amino]-1-methyl-1H-pyrrole-2-carboxamide hydrochloride; and

10. N-{5-[({5-[({5-[({3-[(aminocarbonyl)amino]propyl}amino)carbonyl]-1-methyl-1H-pyrrol-3-yl}amino)carbonyl]-1-methyl-1H-pyrrol-3-yl}amino)carbonyl]-1-methyl-1H-pyrrol-3-yl}-4-[(2-bromoacryloyl)amino]-1-methyl-1H-pyrrole-2-carboxamide.

The above compounds of formula (I), either specifically identified as such or by means of the general formula, are known or easily prepared according to known methods as reported, for instance, in the aforementioned international patent applications WO 90/11277, WO 98/04524, WO 98/21202, WO 99/50265, WO 99/50266 and WO 01/40181.

The present invention further provides a product comprising an acryloyl distamycin derivative of formula (I), as defined above, and an antineoplastic topoisomerase I or II inhibitor, as a combined preparation for simultaneous, separate or sequential use in antitumor therapy.

A further aspect of the present invention is to provide a method of treating a mammal, including humans, suffering from a neoplastic disease state, which method comprises administering to said mammal the above acryloyl distamycin derivative of formula (I) and an antineoplastic topoisomerase I or II inhibitor, in amounts effective to produce a synergistic antineoplastic effect.

The present invention also provides a method for lowering the side effects caused by antineoplastic therapy with an antineoplastic agent in a mammal in need thereof, including humans, the method comprising administering to said mammal a combined preparation comprising an antineoplastic topoisomerase I or II inhibitor and an acryloyl distamycin derivative of formula (I), as defined above, in amounts effective to produce a synergistic antineoplastic effect.

By the term “synergistic antineoplastic effect”, as used herein, it is meant the inhibition of the growth tumor, preferably the complete regression of the tumor, by administering an effective amount of the combination comprising an acryloyl distamycin derivative of formula (I) and a topoisomerase I or II inhibitor to mammals, including humans. By the term “administered” or “administering”, as used herein, it is meant parenteral and/or oral administration; the term “parenteral” means intravenous, subcutaneous and intramuscular administration.

In the method of the present invention, the acryloyl distamycin derivative may be administered simultaneously with the compound having topoisomerase I or II inhibitory activity, for example with a compound of the camptothecin, anthracycline, mitoxanitrone, epipodophyllotoxin, or acridine class. Alternatively, both compounds may be administered sequentially in either order.

In this respect, it will be appreciated that the actual preferred method and order of administration will vary according to, inter alia, the particular formulation of the acryloyl distamycin of formula (I) being used, the particular formulation of the topoisomerase I or II inhibitor being used, for instance the camptothecins such as CPT-11, topotecan, 9-AC; the anthracyclines such as doxorubicin, daunorubicin, epirubicin, idarubicin, nemorubicin; the anthraquinones such as mitoxantrone and losoxantrone; the epipodophyllotoxins such as etoposide, teniposide; the acridine derivatives such as amsacrine and actinomycin D, the particular tumor model being treated as well as the particular host being treated.

To administer the acryloyl distamycin derivative of formula (I), according to the method of the invention, the course of therapy generally employed comprises doses varying from about 0.05 to about 100 mg/m² of body surface area and, more preferably, from about 0.1 to about 50 mg/m² of body surface area. For the administration of the topoisomerase I or II inhibitor, according to the method of the invention, the course of therapy generally employed comprises

when administering camptothecins: doses varying from about 1 to about 1000 mg/m² of body surface area and, more preferably, from about 10 to about 500 mg/m² of body surface area;

when administering anthracyclines: doses varying from about 0.1 to about 1000 mg/m² of body surface area and, more preferably, from about 0.5 to about 500 mg/m² of body surface area;

when administering epipodophyllotoxins: doses varying from about 1 to about 500 mg/m² of body surface area and, more preferably, from about 10 to about 400 mg/m² of body surface area;

when administering anthraquinones: doses varying from about 1 to about 300 mg/m² of body surface area and, more preferably, from about 5 to about 100 mg/m² of body surface area.

when administering acridine and actinomycin D derivatives: doses varying from about 1 to about 1000 mg/m² of body surface area and, more preferably, from about 10 to about 500 mg/m² of body surface area.

The antineoplastic therapy of the present invention is particularly suitable for treating breast, ovary, lung, colon, kidney, stomach, pancreas, liver, melanoma, leukemia and brain tumors in mammals, including humans.

In a further aspect, the present invention is directed to the preparation of a pharmaceutical composition comprising an effective amount of an acryloyl distamycin derivative of formula (I), as defined above, and an antineoplastic topoisomerase I or II inhibitor, in the preparation of a medicament for use in the prevention or treatment of metastasis or in the treatment of tumors by inhibition of angiogenesis.

As stated above, the effect of an acryloyl distamycin derivative of formula (1) and a topoisomerase I or II inhibitor, such as an anthracycline or etoposide derivative, is significantly increased without a parallel increase of toxicity. In other words, the combined therapy of the present invention enhances the antitumoral effects of the acryloyl distamycin derivative and of the topoisomerase I or II inhibitor and, hence, provides the most effective and least toxic treatment for tumors.

The superadditive effects of the combined preparations of the invention are shown, for instance, by the following in vivo tests, which are intended to illustrate the present invention without posing any limitation to it.

Table 1 shows the antileukemic activity on disseminated L1210 murine leukemia obtained by combining N-(5-{[(5-{[(5-{[(2-{[amino(imino)methyl]amino}ethyl)amino]carbonyl}-1-methyl-1H-pyrrol-3-yl)amino]carbonyl}-1-methyl-1H-pyrrol-3-yl)amino]carbonyl}-1-methyl-1H-pyrrol-3-yl)-4-[(2-bromoacryloyl)amino]-1-methyl-1H-pyrrole-2-carboxamide hydrochloride, as a representative compound of formula (I)—internal code PNU 166196, with doxorubicin. At the dose of 10 mg/kg of doxorubicin alone (day +1 after tumor injection and 2 hours after PNU 166196 administration) and at the dose of 0.52 mg/kg of PNU 166196 alone (days +1) were associated, without toxicity, ILS % values of 58 and 33, respectively. Combining doxorubicin and PNU 166196 at the same doses with the same schedule, an increase of activity with ILS % value of 100 was observed, thus indicating a synergistic effect.

TABLE 1
Antileukemic activity against disseminated L1210¹ murine leukemia of an
acryloyl distamycin derivative (I) in combination with doxorubicin

| | Treatment² | Dose | | |
|---|---|---|---|---|
| Compound | schedule | (mg/kg/day) | ILS %³ | Tox⁴ |
| PNU 166196 | iv +1 | 0.52 | 33 | 0/10 |
| Doxorubicin | iv +1(*) | 10 | 58 | 0/10 |
| PNU 166196 + | iv +1 | 0.52 + 10 | 100 | 0/10 |
| Doxorubicin | iv +1(*) | | | |

¹L1210 leukemia cells (10⁵/mouse CD2F1) are injected IV on Day 0.
²Treatment is given IV.
³Increase in life span: [(median survival time of treated mice/median survival time of controls) × 100] − 100.
⁴Number of toxic deaths/number of mice.
(*)doxorubicin was administered 2 h after PNU 166196 administration.

For these experiments, PNU 166196 and doxorubicin were solubilized in water for injection.

1. A pharmaceutical composition comprising a pharmaceutically acceptable carrier or excipient and, as active ingredient,
an acryloyl distamycin derivative of formula (I): 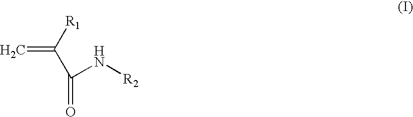
wherein:
R₁ is a bromine or chlorine atom;
R₂ is a distamycin or distamycin-like framework; or a pharmaceutically acceptable salt thereof; and
an antineoplastic topoisomerase inhibitor of type I or II.

2. A pharmaceutical composition according to claim 1 wherein the topoisomerase inhibitor is a topoisomerase II inhibitor selected from anthracycline derivatives, including doxorubicin, daunorubicin, epirubicin, nemorubicin and idarubicin; epipodophyllotoxin compounds including etoposide and teniposide; anthraquinone derivatives including mitoxantrone and losoxantrone; acridine derivatives including amsacrine and dactinomycin.

3. A pharmaceutical composition according to claim 2 wherein the topoisomerase II inhibitor is doxorubicin or etoposide.

4. A pharmaceutical composition according to claim 1 comprising an acryloyl distamycin derivative of formula (I) 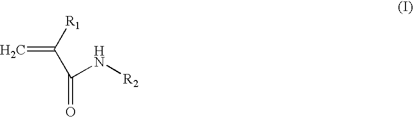
wherein:
R₁ is a bromine or chlorine atom;
R₂ is a group of formula (II) 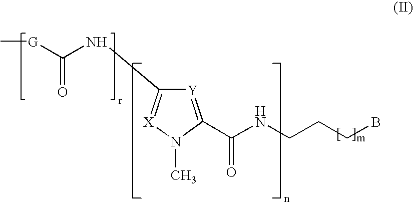
wherein
m is an integer from 0 to 2;
n is an integer from 2 to 5;
r is 0 or 1;
X and Y are, the same or different and independently for each heterocyclic ring, a nitrogen atom or a CH group;
G is phenylene, a 5 or 6 membered saturated or unsaturated heterocyclic ring with from 1 to 3 heteroatoms selected among N, O or S, or it is a group of formula (III) below: 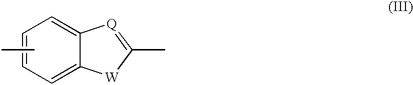
wherein Q is a nitrogen atom or a CH group and W is an oxygen or sulfur atom or it is a group NR₃ wherein R₃ is hydrogen or C₁-C₄ alkyl;
B is selected from the group consisting of 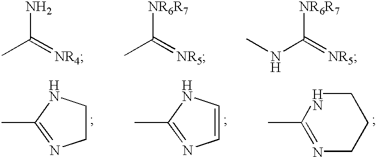
—CN; —NR₅R₆; CONR₅R₆; —NHCONR₅R₆
wherein R₄ is cyano, amino, hydroxy or C₁-C₄ alkoxy; R₅, R₆ and R₇, the same or different, are hydrogen or C₁-C₄ alkyl.

5. A pharmaceutical composition according to claim 4 comprising an acryloyl distamycin derivative of formula (I) wherein R₁, R₂ and B are as defined in claim 4, r is 0, m is 0 or 1 and n is 4.

6. A pharmaceutical composition according to claim 5 comprising an acryloyl distamycin derivative of formula (I) wherein R₁ and R₂ are as defined in claim 4, r is 0, m is 0 or 1, n is 4, X and Y are both CH groups and B is selected from: 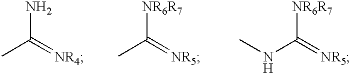
—CN; —CONR₅R₆; —NHCONR₅R₆
wherein R₄ is cyano or hydroxy and R₅, R₆ and R₇, the same or different, are hydrogen or C₁-C₄ alkyl.

7. A pharmaceutical composition according to claim 1 comprising an acryloyl distamycin derivative, optionally in the form of a pharmaceutically acceptable salt, selected from the group consisting of:
1. N-(5-{[(5-{[(5-{[(2-{[amino(imino)methyl]amino}ethyl)amino]carbonyl}-1-methyl-1H-pyrrol-3-yl)amino]carbonyl}-1-methyl-1H-pyrrol-3-yl)amino]carbonyl}-1-methyl-1H-pyrrol-3-yl)-4-[(2-bromoacryloyl)amino]-1-methyl-1H-pyrrole-2-carboxamide hydrochloride;
2. N-(5-{[(5-{[(5-{[(2-{[amino(imino)methyl]amino}propyl)amino]carbonyl}-1-methyl-1H-pyrrol-3-yl)amino]carbonyl}-1-methyl-1H-pyrrol-3-yl)amino]carbonyl}-1-methyl-1H-pyrrol-3-yl)-4-[(2-bromoacryloyl)amino]-1-methyl-1H-pyrrole-2-carboxamide hydrochloride;
3. N-(5-{[(5-{[(5-{[(3-amino-3-iminopropyl)amino]carbonyl}-1-methyl-1H-pyrrol-3-yl)amino]carbonyl}-1-methyl-1H-pyrrol-3-yl)amino]carbonyl}-1-methyl-1H-pyrrol-3-yl)-4-[(2-bromoacryloyl)amino]-1-methyl-1H-pyrrole-2-carboxamide hydrochloride;
4. N-(5-{[(5-{[(5-{[(3-amino-3iminopropyl)amino]carbonyl}-1-methyl-1H-pyrrol-3 yl)amino]carbonyl}-1-methyl-1H-pyrrol-3-yl)amino]carbonyl}-1-1-methyl-1H-pyrrol-3-yl)-4-[(2-bromoacryloyl)amino]-1-methyl-1H-imidazole-2-carboxamide hydrochloride;
5. N-(5-{[(5-{[(5-{[(3-amino-3-iminopropyl)amino]carbonyl}-1-methyl-1H-pyrrol-3-yl)amino]carbonyl}-1-methyl-1H-pyrrol-3-yl)amino]carbonyl}-1-methyl-1H-pyrrol-3-yl)-3-[(2-bromoacryloyl)amino]-1-methyl-1H-pyrazole-5-carboxamide hydrochloride;
6. N-(5-{[(5-{[(5-{[(3-amino-3-oxopropyl)amino]carbonyl}-1-methyl-1H-pyrrol-3-yl)amino]carbonyl}-1-methyl-1H-pyrrol-3-yl)amino]carbonyl}-1-methyl-1H-pyrrol-3-yl)-3-[(2-bromoacryloyl)amino]-1-methyl-1H-pyrazole-5-carboxamide;
7. N-(5-{[(5-{[(5-{[(2-{[amino(imino)methyl]amino}ethyl)amino]carbonyl}-1-methyl-1H-pyrrol-3-yl)amino]carbonyl}-1-methyl-1H-pyrrol-3-yl)amino]carbonyl}-1-methyl-1H-pyrrol-3-yl)-4-[(2-chloroacryloyl)amino]-1-methyl-1H-pyrrole-2-carboxamide hydrochloride;
8. N-(5-{[(5-{[(3-{[amino(imino)methyl]amino}propyl)amino]carbonyl}-1-methyl-1H-pyrrol-3-yl)amino]carbonyl}-1-methyl-1H-pyrrol-3-yl)-4-[(2-bromoacryloyl)amino]-1-methyl-1H-pyrrole-2-carboxamide hydrochloride;
9. N-(5-{[(5-{[(3-amino-3-iminopropyl)amino]carbonyl}-1-methyl-1H-pyrrol-3-yl)amino]carbonyl}-1-methyl-1H-pyrrol-3-yl)-4-[(2-bromoacryloyl)amino]-1-methyl-1H-pyrrole-2-carboxamide hydrochloride; and
10. N-{5-[({5-[({5-[({3-[(aminocarbonyl)amino]propyl}amino)carbonyl]-1-methyl-1H-pyrrol-3-yl}amino)carbonyl]-1-methyl-1H-pyrrol-3-yl}amino)carbonyl]-1-methyl-1H-pyrrol-3-yl}-4-[(2-bromoacryloyl)amino]-1-methyl-1H-pyrrole-2-carboxamide.

8. Products comprising an acryloyl distamycin derivative of formula (I): 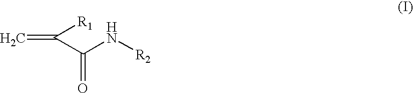
wherein:
R₁ is a bromine or chlorine atom;
R₂ is a distamycin or distamycin-like framework; or a pharmaceutically acceptable salt thereof; and an antineoplastic topoisomerase inhibitor of type I or II, as a combined preparation for simultaneous, separate or sequential use in the treatment of tumors.

9. Products according to claim 8 wherein the topoisomerase inhibitor is a topoisomerase II inhibitor selected from anthracycline derivatives, including doxorubicin, daunorubicin, epirubicin, nemorubicin and idarubicin; epipodophyllotoxin compounds including etoposide and teniposide; anthraquinone derivatives including mitoxantrone and losoxantrone; acridine derivatives including amsacrine and dactinomycin.

10. Products according to claim 9 wherein the topoisomerase II inhibitor is doxorubicin or etoposide.

11. Products according to claim 8 comprising an acryloyl distamycin derivative of formula (I) 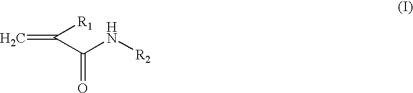
wherein:
R₁ is a bromine or chlorine atom;.
R₂ is a group of formula (II) 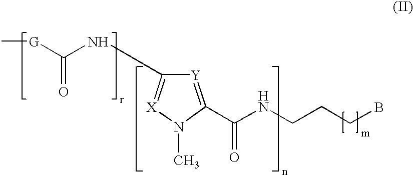
wherein
m is an integer from 0 to 2;
n is an integer from 2 to 5;
r is 0 or 1;
X and Y are, the same or different and independently for each heterocyclic ring, a nitrogen atom or a CH group;
G is phenylene, a 5 or 6 membered saturated or unsaturated heterocyclic ring with from 1 to 3 heteroatoms selected among N, O or S, or it is a group of formula (III) below: 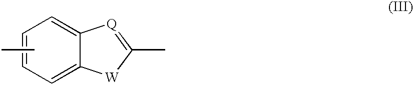
wherein Q is a nitrogen atom or a CH group and W is an oxygen or sulfur atom or it is a group NR₃ wherein R₃ is hydrogen or C₁-C₄ alkyl;
B is selected from the group consisting of 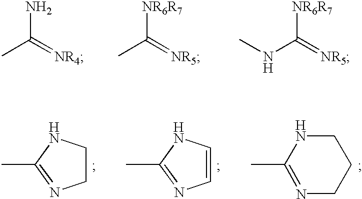
—CN; —NR₅R₆; —CONR₅R₆; —NHCONR₅R₆
wherein R₄ is cyano, amino, hydroxy or C₁-C₄ alkoxy; R₅, R₆ and R₇, the same or different, are hydrogen or C₁-C₄ alkyl.

12. Products according to claim 8 wherein the acryloyl distamycin derivative is selected from the group as defined in claim 7.

13. Use of an acryloyl distamycin derivative of formula (I), as defined in any one of claims from 1 to 7 in the preparation of a medicament for use in combination therapy with an antineoplastic topoisomerase I or II inhibitor in the treatment of tumors.

14. Use according to claim 13 wherein the medicament farther comprises the said topoisomerase I or II inhibitor.

15. Use according to claim 13 or 14 wherein the topoisomerase inhibitor is a topoisomerase II inhibitor selected from etoposide or doxorubicin.

16. Use according to claim 13 or 14 wherein the acryloyl distamycin derivative is selected from the group as defined in claim 7.

17. Use according to any one of claims from 13 to 16 wherein the tumor is selected from breast, ovary, lung, colon, kidney, stomach, pancreas, liver, melanoma, leukemia and brain tumors.

18. Use of an acryloyl distamycin derivative of formula (I), as defined in any one of claims from 1 to 7 in the preparation of a medicament for use in combination therapy with an antineoplastic topoisomerase I or II inhibitor in the prevention or treatment of metastasis or in the treatment of tumors by inhibition of angiogenesis.

19. Use according to claim 18 wherein the medicament further comprises the said topoisomerase I or II inhibitor.

20. A method of treating a mammal, including humans, suffering from a neoplastic disease state, which method comprises administering to said mammal the acryloyl distamycin derivative of formula (I), as defined in any one of claims from 1 to 7, and an antineoplastic topoisomerase I or II inhibitor, in amounts effective to produce a synergistic antineoplastic effect.

21. A method for lowering the side effects caused by antineoplastic therapy with an antineoplastic agent, in a mammal in need thereof including humans, the method comprising administering to said mammal a combined preparation comprising an antineoplastic topoisomerase I or If inhibitor and an acryloyl distamycin derivative of formula (I), as defined in any one of claims from 1 to 7, in amounts effective to produce a synergistic antineoplastic effect.

22. A pharmaceutical composition according to claim 1 wherein the distamycin 30 derivative, optionally in the form of a pharmaceutically acceptable salt, is N-(5-{[(5-{[(5-{[(2-{[amino(imino)methyl]amino}ethyl)amino]carbonyl}-1-methyl-1H-pyrrol-3yl)amino]carbonyl}-1-methyl-1H-pyrrol-3-yl)amino]carbonyl}-1-methyl-1H-pyrrol-3-yl)-4-[(2-bromoacryloyl)amino]-1-methyl-1H-pyrrole-2-carboxamide, and wherein the topoisomerase inhibitor is a topoisomerase II inhibitor selected from the group consisting of etoposide or doxorubicin.
